(19) United States
(12) Patent Application Publication
(10) Pub. No.: US 2023/0170058 A1
(43) Pub. Date: Jun. 1, 2023

(54) SYSTEM AND METHOD FOR ENABLING IN-SILICO PHENOTYPIC SCREENING OF DRUGS

(71) Applicant: Innoplexus AG, Eschborn (DE)

(72) Inventors: Om Sharma, Pune (IN); Irfan Yunus Tamboli, Pune (IN); Werner Seiz, Hofheim (DE)

(73) Assignee: Innoplexus AG, Eschborn (DE)

(21) Appl. No.: 17/539,626

(22) Filed: Dec. 1, 2021

(51) Int. Cl. G16C 20/50; G16C 20/90; G16B 25/10; G16C 20/70

(52) U.S. Cl. CPC G16C 20/50; G16B 25/10; G16C 20/70; G16C 20/90

(57) ABSTRACT

A system for enabling in-silico phenotypic screening of drugs, the system is communicably coupled to a phenotype ontological databank. The system include a processor communicably coupled to a memory. The processor is configured to receive a name of at least one drug as an input, fetch targets of at least one existing drug that is similar to the at least one drug to obtain a drug target list, determine, phenotypes of the at least one drug based on associations between the targets in the drug target list and the phenotypes, said associations being accessed from the phenotype ontological databank, generate a network comprising the at least one drug, the targets and the phenotypes, determine a plurality of groups of similar phenotypes that belong to similar biological processes or clinical pathologies, and determine expressions of the phenotypes in each of a plurality of tissues, based on gene, protein and tissue expression data accessed from at least one database, determine tissues where the phenotypes of a given group are relevant and diseases associated with the tissues, based on said expressions of the phenotypes, thereby enabling phenotypic screening of the at least one drug.

### TECHNICAL FIELD

The present disclosure relates generally to screening of drugs; and more specifically, to in-silico phenotypic screening of drugs.

### BACKGROUND

Conventionally, the pharmaceutical industry has been dependent on a target-based approach to carry out screening of known or unknown drugs. Herein, the target-based approach is a rational approach for screening drugs targeting a biomolecule that causes diseases. Observationally, target-based approach uses multiple in-silico and computational methods and is more successful in identification of follow-up drugs. However, the target-based approach provides two major setbacks of target deconvolution and polypharmacology. Due to the recurring setbacks, the pharmaceutical industry is now leaning towards a phenotypic-based approach for the screening of drugs.

Phenotypic screening based on in-vitro methods is highly time consuming and resource extensive. Herein, modern robotic high throughput screening (HTS) platforms enables the screening of within a chemical library comprising tens of thousands of drugs at a time for a single phenotype. However, the screening of multiple phenotypes in parallel phenotypic assays still has its limitations even with the use of the HTS platforms. Therefore, due to the practical constraints of knowing polypharmacology of the drugs and/or compounds is a highly inefficient process using an in-vitro approach. Moreover, shortlisting of phenotypes to be screened brings in researcher bias and may lead to exclusion of important phenotypes influenced by the drug. Furthermore, screening of the phenotypes using traditional experimental approach might take years to accomplish even a part of it. Additionally, simultaneous screening of cellular, molecular and clinical phenotype is not possible currently.

There are screening methods available to screen for multi-target drugs and/or drug combinations which comprises showing a drug target pair relevant to a particular disease. However, the screening method is not able to simultaneously screen the cellular and the clinical phenotype similar to the molecular phenotype. Furthermore, multiple targets of drug may be determined with the help of random forest quantitative structure-activity relationship (QSAR) models. However, the screening method is not able to simultaneously screen the cellular and the clinical phenotype similar to the molecular phenotype.

Therefore, in the light of the foregoing discussion, there still exists a need to overcome the aforementioned drawbacks associated with known techniques for screening of drugs.

### SUMMARY

The present disclosure seeks to provide a system for enabling in-silico phenotypic screening of drugs. The present disclosure also seeks to provide a method for enabling in-silico phenotypic screening of drugs. An aim of the present disclosure is to provide a solution that overcomes at least partially the problems encountered in prior art.

In one aspect, the present disclosure provides a system for enabling in-silico phenotypic screening of drugs, the system is communicably coupled to
a phenotype ontological databank comprising information pertaining to a plurality of drugs and phenotypes corresponding to each of the plurality of drugs thereof;
   wherein the system comprises a processor communicably coupled to a memory, the processor configured to
receive a name of at least one drug as an input;
fetch targets of at least one existing drug that is similar to the at least one drug to obtain a drug target list;
determine, phenotypes of the at least one drug based on associations between the targets in the drug target list and the phenotypes, said associations being accessed from the phenotype ontological databank;
   phenotype- generate a network comprising the at least one drug, the targets and the phenotypes;
determine a plurality of groups of similar phenotypes that belong to similar biological processes or clinical pathologies; ; and
determine expressions of the phenotypes in each of a plurality of tissues, based on gene, protein and tissue expression data accessed from at least one database;
determine tissues where the phenotypes of a given group are relevant and diseases associated with the tissues, based on said expressions of the phenotypes, thereby enabling phenotypic screening of the at least one drug.

In another aspect, the present disclosure provides a computer-implemented method for enabling in-silico phenotypic screening of drugs, wherein the method is implemented using a system communicably coupled to a phenotype ontological databank comprising information pertaining to a plurality of drugs and phenotypes corresponding to each of the plurality of drugs thereof, the method comprising:
receiving a name of at least one drug as an input;
fetching targets of at least one existing drug that is similar to the at least one drug to obtain a drug target list;
determining, phenotypes of the at least one drug based on associations between the targets in the drug target list and the phenotypes, said associations being accessed from the phenotype ontological databank;
generating a network comprising the at least one drug, the targets and the phenotypes;
determining a plurality of groups of similar phenotypes that belong to similar biological processes or clinical pathologies;
determining expressions of the phenotypes in each of a plurality of tissues, based on gene, protein and tissue expression data accessed from at least one database; and
determining tissues where the phenotypes of a given group are relevant and diseases associated with the tissues, based on said expressions of the phenotypes, thereby enabling phenotypic screening of the at least one drug.

Embodiments of the present disclosure substantially eliminate or at least partially address the aforementioned problems in the prior art, and enable efficient in-silico screening of drugs.

Additional aspects, advantages, features and objects of the present disclosure would be made apparent from the drawings and the detailed description of the illustrative embodiments construed in conjunction with the appended claims that follow.

It will be appreciated that features of the present disclosure are susceptible to being combined in various combinations without departing from the scope of the present disclosure as defined by the appended claims.

### BRIEF DESCRIPTION OF THE DRAWINGS

The summary above, as well as the following detailed description of illustrative embodiments, is better understood when read in conjunction with the appended drawings. For the purpose of illustrating the present disclosure, exemplary constructions of the disclosure are shown in the drawings. However, the present disclosure is not limited to specific methods and instrumentalities disclosed herein. Moreover, those skilled in the art will understand that the drawings are not to scale. Wherever possible, like elements have been indicated by identical numbers.

Embodiments of the present disclosure will now be described, by way of example only, with reference to the following diagrams wherein:

FIG. 1 is a block diagram of a system for enabling in-silico phenotypic screening of drugs, in accordance with an embodiment of the present disclosure;

FIG. 2 is input of at least one drug, in accordance with the implementation of the present disclosure;

FIG. 3 is a system for identifying targets of at least one drug to obtain a drug target list, in accordance with the implementation of the present disclosure;

FIG. 4 is a system for determining phenotypes of at least one drug, in accordance with the implementation of the present disclosure;

FIG. 5 is a Drug-Target-Phenotype (DTP) network, in accordance with the embodiments of the present disclosure;

FIG. 6 is a visual representation to determine a parent level phenotype, in accordance with the embodiments of the present disclosure;

FIGS. 7A, 7B and 7C collectively shows a visual representation of phenotypic signatures for a plurality of drugs, in accordance with the embodiments of the present disclosure; and

FIGS. 8A and 8B collectively illustrate a flowchart depicting steps of a method for enabling in-silico phenotypic screening of drugs, in accordance with the embodiments of the present disclosure.

In the accompanying drawings, an underlined number is employed to represent an item over which the underlined number is positioned or an item to which the underlined number is adjacent. A non-underlined number relates to an item identified by a line linking the non-underlined number to the item. When a number is non-underlined and accompanied by an associated arrow, the non-underlined number is used to identify a general item at which the arrow is pointing.

### DETAILED DESCRIPTION OF EMBODIMENTS

In one aspect, the present disclosure provides a system for enabling in-silico phenotypic screening of drugs, the system is communicably coupled to
a phenotype ontological databank comprising information pertaining to a plurality of drugs and phenotypes corresponding to each of the plurality of drugs thereof;
   wherein the system comprises a processor communicably coupled to a memory, the processor configured to
receive a name of at least one drug as an input;
fetch targets of at least one existing drug that is similar to the at least one drug to obtain a drug target list;
determine, phenotypes of the at least one drug based on associations between the targets in the drug target list and the phenotypes, said associations being accessed from the phenotype ontological databank;
   phenotype- generate a network comprising the at least one drug, the targets and the phenotypes;
determine a plurality of groups of similar phenotypes that belong to similar biological processes or clinical pathologies;
determine expressions of the phenotypes in each of a plurality of tissues, based on gene, protein and tissue expression data accessed from at least one database; and
determine tissues where the phenotypes of a given group are relevant and diseases associated with the tissues, based on said expressions of the phenotypes, thereby enabling phenotypic screening of the at least one drug.

In another aspect, the present disclosure provides a computer-implemented method for enabling in-silico phenotypic screening of drugs, wherein the method is implemented using a system communicably coupled to a phenotype ontological databank comprising information pertaining to a plurality of drugs and phenotypes corresponding to each of the plurality of drugs thereof, the method comprising:
receiving a name of at least one drug as an input;
fetching targets of at least one existing drug that is similar to the at least one drug to obtain a drug target list;
determining, phenotypes of the at least one drug based on associations between the targets in the drug target list and the phenotypes, said associations being accessed from the phenotype ontological databank;
generating a network comprising the at least one drug, the targets and the phenotypes;
determining a plurality of groups of similar phenotypes that belong to similar biological processes or clinical pathologies;
determining expressions of the phenotypes in each of a plurality of tissues, based on gene, protein and tissue expression data accessed from at least one database;
determining tissues where the phenotypes of a given group are relevant and diseases associated with the tissues, based on said expressions of the phenotypes, thereby enabling phenotypic screening of the at least one drug.

The following detailed description illustrates embodiments of the present disclosure and ways in which they can be implemented. Although some modes of carrying out the present disclosure have been disclosed, those skilled in the art would recognize that other embodiments for carrying out or practising the present disclosure are also possible.

The system and method of the present disclosure aims to provide an efficient in-silico platform for phenotypic screening drugs, wherein the in-silico platform is able to screen chemical libraries to screen phenotypes of the at least one drug based on associations between the targets in the drug target list and the phenotypes. Furthermore, the shortlisting of phenotypes to be screened is performed in-vitro, hence there is no researcher bias, wherein the research bias usually leads to exclusion of important phenotypes influenced by the drug. Additionally, the in-silico platform enables virtual screening of the phenotypes within a few hours, thereby saving in terms of cost and time. Notably, the in-silico platform also provides prediction of diverse cellular and clinical phenotypes along with molecular phenotypes. Furthermore, the in-silico platform supports in-vitro phenotypic drug discovery (PDD) analysis for a range of phenotypic assays and phenotypes.

The system comprises a phenotype ontological databank comprising information pertaining to a plurality of drugs and phenotypes corresponding to each of the plurality of drugs thereof. Herein, the phenotype ontological databank uses ontology, which is a data model that represents concepts, attributes, and relationships in the form of a directed acyclic graph. Furthermore, the phenotype ontological databank provides exploratory analysis of microarray and other forms of high-throughput data. Additionally, the phenotype ontological databank is created with the purpose of covering all phenotypes corresponding to the plurality of drugsphenotype. phenotypeMoreover, each of the plurality of drugs may have multiple phenotypes.

Throughout the present disclosure, the term “processor” refers to a computational element that is operable to respond to and processes instructions that drive the system. Furthermore, the term “processor” may refer to one or more individual processors, processing devices and various elements associated with a processing device that may be shared by other processing devices. Additionally, the one or more individual processors, processing devices and elements are arranged in various architectures for responding to and processing the instructions that drive the system.

Throughout the present disclosure, the term “memory” refers to a volatile or persistent medium, such as an electrical circuit, magnetic disk, virtual memory or optical disk, in which a computer can store data or software for any duration. Optionally, the memory is a non-volatile mass storage such as physical storage media. Furthermore, a single memory may encompass and, in a scenario, wherein the system is distributed, the processing, memory and/or storage capability may be distributed as well.

The system comprises the processor communicably coupled to the memory, wherein the processor is configured to receive a name of at least one drug as an input. Herein, a name of the at least one drug such as “Pazopanib”, as a Simplified Molecular Input Line Entry System (SMILE), wherein SMILES is a chemical line notation that allows a user to represent a chemical structure in a way that can be used by the processor. Furthermore, the name of at least one drug as an input may be in the form of a two-dimensional (2D) chemical structure or a three-dimensional (3D) chemical structure, a compound list, wherein the compounds of the at least one drug may be organized, such as for example, according to a list of inorganic compounds and/or a list of biomolecules. Additionally, the input of the name of the at least one drug may be in the form of a chemical library, wherein a chemical library is a collection of different real stored chemicals and/or virtual chemical compounds containing relevant information, such as for example, but not limited to, chemical structure, purity, quantity and physiochemical characteristics every compound. Typically, the chemical libraries comprise 2D or 3D representation of the chemical compounds as well that are used for computational methods and in-silico screening. Herein, the term “in-silico” refers to biological experiments conducted on a computer or via computer simulation.

The processor is configured to fetch targets of at least one existing drug that is similar to the at least one drug to obtain a target list. Herein, the targets of the at least one existing drug is usually a protein, which is intrinsically associated with a particular disease process.. Herein, the target is identified and characterized by identifying function of a possible therapeutic agent, wherein the therapeutic agent may be a gene and/or protein and their role in a disease. In this regard, the at least one existing drug interacts with multiple targets rather than with a single target. Subsequently, the targets that are identified are listed down in a target list, wherein the target list comprises all the targets relevant to the at least one existing drug that is similar to the at least one drug given as input to the processor.

Optionally, literature mining is used to fetch the targets of the at least one existing drug that is similar to the at least one drug. Herein, a majority of new targets are derived from novel biological discoveries first appearing in scientific literature. Herein, sentences are extracted from publications or documents. Furthermore, literature mining uses various keyword mechanisms and countless forms of indexing or document and/or publication classification, as well as straightforward semantic or text search, wherein sets of documents may be retrieved with the help of literature mining, generally with additional refinements such as Boolean combinations of search terms, iterative refinement of searches and so forth, to obtain the majority of new targets. Herein, certain techniques, such as for example, but not limited to, Name Entity Recognition (NER) may be used on scientific literature to identify chemicals, targets, genes, pathways, diseases and utilized with algorithms to procure additional biologically significant words. Thereafter, a plethora of similarity and partitional clustering techniques may be used to group the majority of new targets based on their common terms.

Optionally, chemical similarity algorithm is used to identify the at least one existing drug that is similar to the at least one drug. Typically, chemical similarity algorithm is an important methodology used to identify compounds with similar bioactivities based on structural similarity between any two drugs. Herein, the fundamental principle behind the chemical similarity algorithm is the chemical similarity principle, which states that if two molecules share similar structures, then they will likely have similar bioactivities. Furthermore, the chemical similarity algorithm most commonly uses approaches that use chemical substructure fingerprints, such as non-hashed structural fingerprints, chemical hashed fingerprints. Typically, in non-hashed structural fingerprints such as Open Babel FP3, each molecule is converted into a binary series of ‘0’ and ‘1’, wherein ‘0’ indicates absence of a particular structure and ‘1’ indicates the presence of the particular substance, so as to compare the chemical similarity between two molecules. Conversely, in chemical hashed fingerprints such as Open Babel FP2, path information is derived from molecular graphs to compare the chemical structures. Thereafter, the chemical similarity is obtained using a distance metric, for instance Tanimoto index and so forth, after procuring chemical fingerprints of the molecules. Moreover, the targets of the at least one drug may be inferred from structured databases with annotated targets sharing highest similarity to the target. Herein, the structured databases may be public bioactivity databases such as for example, but not limited to chemical database maintained by European Bioinformatics Institute of the European Molecular Biology Laboratory (ChEMBL®), PubChem®, DrugBank.

Optionally, machine learning algorithm is used to predict targets of the at least one drug to obtain the drug target list, based on the targets of the at least one existing drug that is similar to the at least one drug. Typically, the machine learning algorithm is a computational approach which can leverage the growing number of large-scale human genomics and proteomics data sets to make in-silico target identification. Herein, machine learning algorithm is used to prioritize the targets according to their similarity to approved drug targets. Notably, the machine learning algorithm predicts the targets of the at least one drug, wherein the at least one drug is an unknown compound. Furthermore, training dataset of the machine learning algorithm may comprise 37,000 compounds and 3000 target information.

Optionally, molecular docking method is used to predict targets of the at least one drug to obtain the drug target list. Herein, molecular docking method is bioinformatic modelling that involves interaction of two or more molecules to provide a stable adduct, wherein the term “adduct” refers to a complex that forms when a chemical binds to a biological molecule, such as protein. Subsequently, the molecular docking depends upon binding properties of the targets and ligands of the at least one drug that is unknown and predicts the 3D structure of any complex. Herein, the molecular docking unstructured databases to search for targets, wherein the targets should be in a proper Protein Databank Format (PDB) format. Additionally, the ligand is prepared as a PDB file using software such as Discovery Studio®. Thereby, the ligands are able to organize based upon their ability to interact with given target. Moreover, the molecular docking of small molecules of small molecules to the targets include a pre-defined sampling of possible conformation of the ligand in a particular groove of the targets so to establish an optimized conformation of the complex. Typically, the molecular docking is performed by simulation approach and shape complementarity approach. In particular, high-throughput virtual screening (HTVS) is used for docking many ligands against one or a few receptors, and a combination of pose identification and scoring algorithms constitute foundation of docking engines, including DOCK and AutoDOCK. Furthermore, results of the molecular docking results are evaluated either by visual inspection of the ligand or quantitatively using a scoring algorithm. Herein, HTVS reduces number of intermediate conformations throughout the process of molecular docking, and also reduces thoroughness of final torsional refinement and sampling.

The processor is configured to determine phenotypes of the at least one drug based on associations between the targets in the drug target list and the phenotypes, said associations being accessed from the phenotype ontological databank. Herein, phenotype screening is useful to screen the at least one drug, wherein the at least one drug may be a first-in-class drug, as there is lack of bias while identifying mechanism of action (MOA) of the at least one drug when it is a first-in-class drug. Furthermore, a physiologically relevant biological system or cellular signaling pathway is directly interrogated by chemical matter to identify biologically active compounds. phenotype. Herein, a database such as Innoplexus® Phenotype Ontology Database may be used, wherein the database comprises data from publicly available structured databases such as QuickGo®, Gene Ontology, Human Phenotype Ontology (HPO), Monarch Initiative and so forth. Furthermore, ontologies of phenotypes of the at least one drug are taken from datasets of QuickGo®, Gene Ontology, Human Phenotype Ontology (HPO). Additionally, association of diseases to the phenotypes are brought in from Monarch Initiative, MalaCards® as well as from unstructured data sources obtained using literature mining. Importantly, the phenotypic ontological databank stores data about ontology of the phenotypes, associated phenotypes of the at least one drug and the association of the disease with the phenotype.

phenotypephenotypephenotypephenotypeThe processor is configured to generate a network comprising the at least one drug, the targets and the phenotypes, namely a Drug-Target-Phenotype (DTP) network. Herein, the DTP network comprises direct and indirect relation of the at least one drug with the phenotypes and phenotypes of the at least one drug. Furthermore, the DTP network are visually represented as simple graphs, with nodes and vertices denoting the at least one drug, the phenotypes of the at least one drug and the phenotypes of the at least one drug, and the links or edges denoting the interactions between them. Additionally, the nodes of the DTP network have a number of edges attached to it, wherein the nodes which has maximum number of edges linked to it are important for the integrity of the network. Moreover, the DTP network are modular in nature, wherein a module comprises a set of nodes that are more densely connected with each other than with other nodes in the network.

Optionally, the processor is configured to determine the phenotypes of the at least one drug by selecting only those phenotypes which are associated with at least two targets from the drug target list.

Optionally, the processor is configured to use statistical methods to prioritize the phenotypes. Herein, the phenotypes are prioritized to gain more confidence in the predicted phenotypes in the DTP network, wherein phenotypes only with a minimum of two overlapping phenotypes from the structured and/or unstructured database is considered. Herein, statistical methods such as t-test and false discovery rate (FDR) may be used, wherein t-test is a type of inferential statistic used to determine if there is a significant difference between the phenotypes, which may be related in certain features, and false discovery rate, is a metric for global confidence assessment of the overlapping phenotypes of the at least one drug. Herein, FDR may be Benjamini and Hochberg (BH FDR) or Bonferroni correction. Typically, BH FDR are used when multiple hypothesis testing is performed and decreases the FDR, and Bonferroni correction is a multiple-comparison correction metric which is used when several dependent or independent statistical tests are being performed simultaneously. Furthermore, the drug target list and their respective phenotypes may be sorted based on perturbation value (p-value). Herein, the p-value is a probability of overlap seen between the drug target and the phenotype. Herein, in case the p-value is lower than a cut-off value, that may be for example, ‘0.005’, the overlap is more significant. Herein, logarithmic value of the p-value to the base 10, or ‘log₁₀p’represents level of significance of each association of the phenotypes.

In an embodiment, the targets of the at least one drug to obtain the drug target list are identified using literature mining, wherein literature mining comprises chemical similarity algorithm, machine learning algorithm and molecular docking. Subsequently, the phenotypes of the at least one drug is determined using the Innoplexus® Phenotype Ontology Database and compared with the drug target list to identify the plurality of overlapping targets therebetween. Consequently, the DTP network using the plurality of overlapping targets. In an example, molecular phenotypes of the at least one drug such as “Duvelisib” is determined, wherein the molecular phenotypes possess an identification code, and the overlapping of phenotypes of “Duvelisib” with the molecular phenotypes is determined along with an overlap count. Subsequently, the structure is shown in Table 1

TABLE 1

| MOLECULAR PHENOTYPE | IDENTIFICATIO N CODE | OVERLAPPING PHENOTYPES | OVERLAP COUNT | P- VALUE |
|---|---|---|---|---|
| Kinase activity | GO:0016301 | [‘PIK3CD’, ‘CCL3’, ‘MTOR’, ‘AKT1’, ‘PIK3CA’] | 5 | 3.83E-07 |
| DNA-binding transcription factor activity | GO:0003700 | [‘TP53’, ‘MYC’, ‘ETS1’, ‘SPI1’, ‘FOXP3’, ‘STAT3’, ‘SMAD2’, ‘FOXO1’] | 8 | 5.35E-07 |
| BH3 domain binding | GO:0051434 | [‘MCL1’, ‘BCL2’, ‘BCL2L1’] | 3 | 1.68E-06 |
| Ubiquitin protein ligase binding | GO:0031625 | [‘TP53’, ‘CD40’, ‘BCL2’, ‘HDAC6’, ‘CXCR4’, ‘HSPA5’, ‘SMAD2’, ‘FOXO1’] | 8 | 1.11E-05 |
| RNA polymerase II cis-regulatory | GO:0000978 | [‘TP53’, ‘HDAC1’, ‘MYC’, ‘ETS1’, | 8 | 1.75E-05 |
| region sequence-Specific DNA binding | | ‘HDAC6’, ‘SPI1’, ‘STAT3’, ‘SMAD2’] | | |
| Core promoter sequence-specific DNA binding | GO:0001046 | [‘HDAC1’, ‘TP53’, ‘MYC’] | 3 | 1.80E-05 |
| Protein serine/threoni ne kinase activity | GO:0004674 | [‘MTOR’, ‘TBK1’, ‘AURKA’, ‘AURKB’, ‘AKT1’, ‘IRAK4’, ‘SYK’] | 7 | 2.46E-05 |
| Protein kinase activity | GO:0004672 | [‘CCL3’, ‘MTOR’, ‘AURKA’, ‘IRAK4’, ‘SYK’] | 5 | 5.87E-05 |
| CCR5 chemokine receptor binding | GO:0031730 | [‘CCL4’, ‘CCL3’] | 2 | 0.00011 6 |
| Activating transcription factor binding | GO:0033613 | [‘HDAC1’, ‘MYC’, ‘SMAD2’] | 3 | 0.00017 8 |
| CCR1 chemokine receptor binding | GO:0031726 | [‘CCL4’, ‘CCL3’] | 2 | 0.00019 3 |
| Repressing transcription factor binding | GO:0070491 | [‘HDAC1’, ‘MYC’, ‘BCL2’] | 3 | 0.00020 1 |
| Transcription factor binding | GO:0008134 | [‘TP53’, ‘HDAC1’, ‘MYC’, ‘SPI1’, ‘STAT3’, ‘SMAD2’] | 6 | 0.00020 4 |
| Integrin binding | GO:0005178 | [‘CD81’, ‘CD40LG’, ‘SYK’, ‘CXCL12’] | 4 | 0.00029 7 |
| Histone deacetylase binding | GO:0042826 | [‘TP53’, ‘HDAC1’, ‘FOXP3’, ‘HDAC6’] | 4 | 0.00038 1 |
| Identical protein binding | GO:0042802 | [‘TP53’, ‘BTK’, ‘PTEN’, ‘CCL3’, ‘BCL2’, ‘CDA’, ‘MTOR’, ‘ETS1’, ‘AKT1’, ‘CXCR4’, ‘CCL4’, ‘BCL2L1’, ‘STAT3’, ‘CD53’] | 14 | 0.00061 6 |
| Protein tyrosine kinase activity | GO:0004713 | [‘BTK’, ‘TNK1’, ‘SYK’] | 3 | 0.00094 5 |
| Protein deacetylase activity | GO:0033558 | [‘HDAC1’, ‘HDAC6’] | 2 | 0.00103 6 |
| Misfolded protein binding | GO:0051787 | [‘HSPA5’, ‘HDAC6’] | 2 | 0.00123 7 |

The processor is configured to determine a plurality of groups of similar phenotypes that belong to similar biological processes or clinical pathologiesphenotype. Furthermore, the molecular phenotype refer to direct effect of a variant at a molecular level such as, changes in gene expression, loss of protein stability and so forth, the cellular phenotype refers to a conglomerate of multiple cellular processes involving gene and protein expression that result in elaboration of a particular morphology and function of a cell, and clinical phenotype refers to observable characteristics or traits of an organism like a human, such as morphology, development, biochemical, phenology and so forth. Furthermore, if the predefined threshold is given to be ‘0.5’ for the given target cluster, then only those targets of the molecular phenotype, or the cellular phenotype or the clinical phenotype will be considered in the target cluster which will possess a similarity score higher than ‘0.5’.

The processor is configured to determine expressions of the phenotypes in each of a plurality of tissues, based on gene, protein and tissue expression data accessed from at least one database. Moreover, the processor is further configured to determine tissues where the phenotypes of a given group are relevant and diseases associated with the tissues, based on said expressions of the phenotypes, thereby enabling phenotypic screening of the at least one drug.

Optionally, the processor is configured to determine a corresponding weightage of each phenotype in a given group and a cumulative weightage of the phenotypes of the given group; and generate a visual representation of the given group based on a ratio of individual weightages of the phenotypes of the given group to the cumulative weightage. Herein, the phenotypes are organized based on differed ontology levels. Furthermore, the weightage of each phenotypes present in the plurality of target clusters is added to generate a visual representation of the parent level phenotype that generates the phenotypic signatures proportional to a cumulative score.

Optionally, the processor is configured to determine the phenotypes of the at least one drug by selecting only those phenotypes which are associated with at least two targets from the drug target list.

In an embodiment, the visual representation may be generated in the form of ontology tree, pie charts, sunburst charts and so forth. Herein, the ontology tree is a network of all ontology terms related to the phenotypes or genes, where the level of output node along with other nodes in the network are visualized. Furthermore, other terms adjacent to the phenotypes or genes and depth of the nodes may also be visualized. Typically, hierarchy of the phenotype comprises sub-paths, wherein the sub-paths comprise different levels for each of the sub-paths, and may go as high as ‘12’. Herein, a simpler view of ontologies of output phenotypes is generated that highlight only major nodes in each path. For instance, for a drug, such as, “Erlotinib”, molecular function is divided into sub-paths, wherein the sub-paths may be “binding” and “catalytic activity”. Subsequently, the sub-path of “catalytic activity” may comprise different levels, such as “hydrolase activity”, “oxidoreductase activity” and so forth. Additionally, the pie charts are used to visualize the top classes of the phenotypes associated with the input. Herein, top phenotypes are from different branches of the ontology tree, thereby providing a broader idea about the major phenotypic classes hit by the input. Moreover, the sunburst chart provides a visual representation of all the major phenotype hits and their weightage. Herein, the sunburst chart is a three-dimensional (3D) chart that also incorporates the phenotype ontology information, which can be viewed by clicking on any of the phenotypes to see next level phenotypes and weightages.

In an embodiment, there may be 11 phenotypes namely, “Target A”, “Target B”, “Target C”, “Target D”, “Target E”, “Target F”, “Target G”, “Target H”, “Target I”, “Target J”, “Target K”. Furthermore, the phenotypes are grouped together in phenotype groups namely, “Pheno_A”, “Pheno_B”, “Pheno_C”, “Pheno_D”, “Pheno_E”, “Pheno_F”. Additionally, there may be two parent level phenotypes namely, “Pheno_1” and “Pheno_2”. Herein the “Pheno_1” and “Pheno_2” comprises the phenotype groups, which further comprises a combination of phenotypes. Notably, the combination of the phenotypes may not be same for the same phenotype group, in case the same phenotype group belongs to a different parent level phenotype. For instance, the parent level phenotype “Pheno_1” comprises the phenotype group “Pheno_A”, wherein “Pheno_A” comprises the phenotypes “Target A” and “Target B”. However, the parent level phenotype “Pheno_2” comprises the phenotype group “Pheno_A”, wherein “Pheno_A” comprises the phenotypes “Target D” and “Target E”. Subsequently, the phenotype group comprises cumulative weightage for the phenotypes. Consequently, the weightage of the phenotype groups are cumulated to provide weightage of the parent level phenotype. Subsequently, a structure similar to Table 2 is observed

TABLE 2

| S. No. | Parent level phenotype | Phenotype Group | Phenotypes | Phenotype weightages | Group weightage |
|---|---|---|---|---|---|
| 1 | Pheno_1 | Pheno_A | Target A, Target B | 0.3 | 1.9 |
| | | Pheno_B | Target B, Target C, Target E | 0.4 | |
| | | Pheno_C | Target A, Target B, Target C | 0.5 | |
| | | Pheno_D | Target E, Target F | 0.7 | |
| 2 | Pheno_2 | Pheno_A | Target D, Target E | 0.2 | 1.5 |
| | | Pheno_B | Target F, Target G, Target H | 0.5 | |
| | | Pheno_C | Target G, Target H, Target I | 0.1 | |
| | | Pheno_D | Target B, Target C, Target F | 0.2 | |
| | | Pheno_E | Target J, Target K | 0.1 | |
| | | Pheno_F | Target J, Target K, Target B | 0.4 | |

In an embodiment, the processor is configured to prioritize tissues and disease association by assessing expression of the phenotypes. Herein, data to assess the association of the phenotypes in various tissues is procured from a database such as Human Protein Atlas and/or comprehensive transcriptome datasets, such as Genotype-tissue Expression Project (GTEx), wherein GTEx is a large-scale effort where deoxyribonucleic acid (DNA) and ribonucleic acid (RNA) were collected from multiple tissue samples thereby providing a comprehensive cross-tissue survey of functional consequences of genetic variation. Subsequently, the tissues and the disease association after assessing expression of the phenotypes may be visually represented. Furthermore, a graph may be generated which visually represents the overlap of the tissue or the disease association with the phenotype. For instance, phenotype such as “kinase activity” is checked for the overlap in the following tissues such as, “adipose tissue”, “adrenal gland”, “appendix”, “bone marrow” and so forth.

The present disclosure also relates to the method as described above. Various embodiments and variants disclosed above apply mutatis mutandis to the method.

Optionally, the method comprises: determining a corresponding weightage of each phenotype in a given group and a cumulative weightage of the phenotypes of the given group; and generating a visual representation of the given group based on a ratio of individual weightages of the phenotypes of the given group to the cumulative weightage.

Optionally, the method comprises using literature mining to fetch the targets of the at least one existing drug that is similar to the at least one drug.

Optionally, the method comprises using a chemical similarity algorithm to identify the at least one existing drug that is similar to the at least one drug.

In an embodiment, the method comprises using a machine learning algorithm to predict targets of the at least one drug to obtain the drug target list, based on the targets of the at least one existing drug that is similar to the at least one drug.

In an embodiment, the method further comprises using a molecular docking method to predict targets of the at least one drug to obtain the drug target list.

In an embodiment, the method further comprises determining the phenotypes of the at least one drug by selecting only those phenotypes which are associated with at least two targets from the drug target list.

### DETAILED DESCRIPTION OF THE DRAWINGS

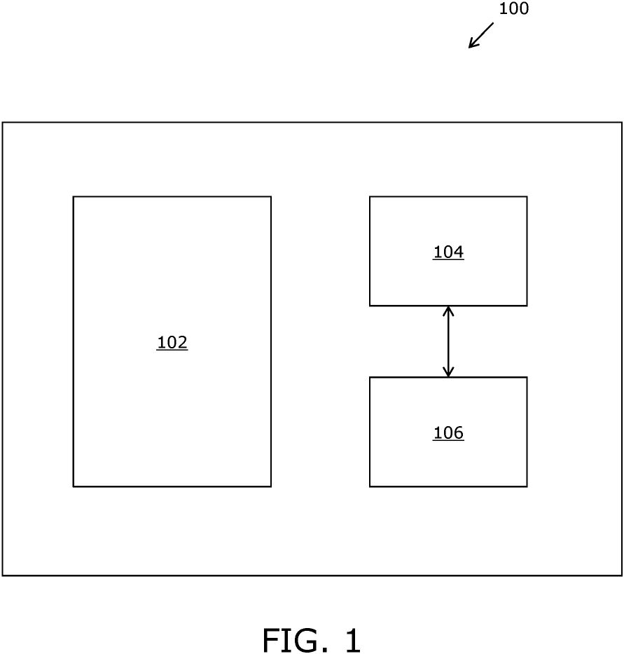

Referring to FIG. 1, there is shown a block diagram of a system 100 for enabling in-silico phenotypic screening of drugs, in accordance with the implementation of the present disclosure. Herein, the system 100 comprises a phenotype ontological databank 102, wherein the phenotype ontological databank 102 comprises information pertaining to a plurality of drugs and phenotypes corresponding to each of the plurality of drugs thereof. Furthermore, the system 100 comprises a processor 104 communicably coupled to a memory 106.

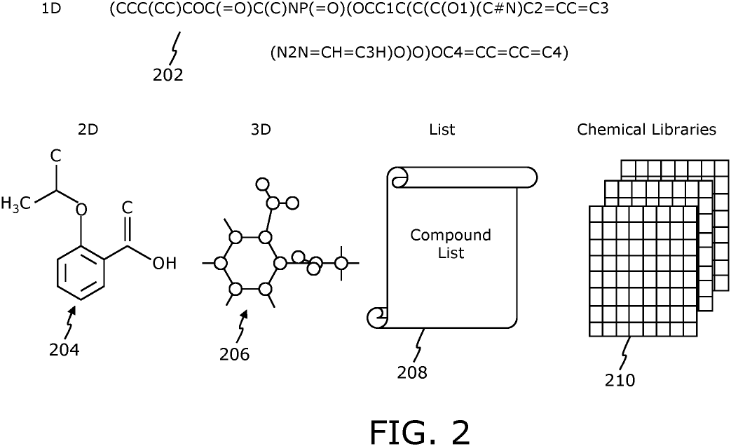

Referring to FIG. 2, there is shown input of name of at least one drug as an input, in accordance with the implementation of the present disclosure. Herein, the input may be in the form of a Simplified Molecular Input Line Entry System (SMILE) 202, wherein SMILES is a chemical line notation that allows a user to represent a chemical structure. Furthermore, the input may be in the form of a two-dimensional (2D) chemical structure 204 or a three dimensional (3D) chemical structure 206, a compound list 208 or as a chemical library 210.

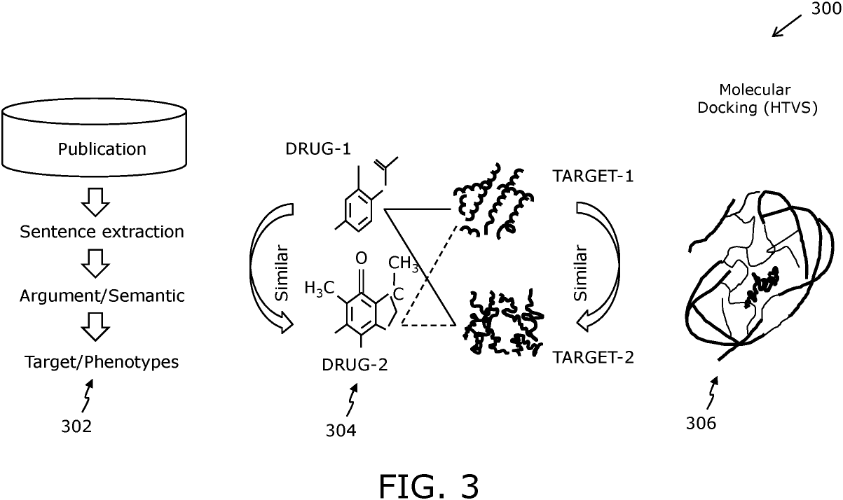

Referring to FIG. 3, there is shown a system 300 for fetching targets of at least one existing drug that is similar to the at least one drug to obtain a drug target list, in accordance with the implementation of the present disclosure. Herein, literature mining 302 is used to fetch targets of the at least one existing drug from publications. Subsequently, sentence extraction is executed using semantic search and consequently the targets of the at least one existing drug is identified. Optionally, chemical similarity algorithm 304 is used to identify targets of the at least one existing drug. Herein, the chemical similarity algorithm 304 identifies compounds with similar bioactivities based on structural similarity between a first drug “Drug 1” and a second drug “Drug 2”. Additionally, targets such as “Target 1” and “Target 2” may be inferred with annotated targets sharing highest similarity. Optionally, molecular docking method 306 is used to fetch targets of the at least one drug that is unknown. Herein, molecular docking method 306 is a bioinformatic modelling that involves interaction of two or more molecules to provide a stable adduct, wherein the term “adduct” refers to a complex that forms when a chemical binds to a biological molecule, such as protein.

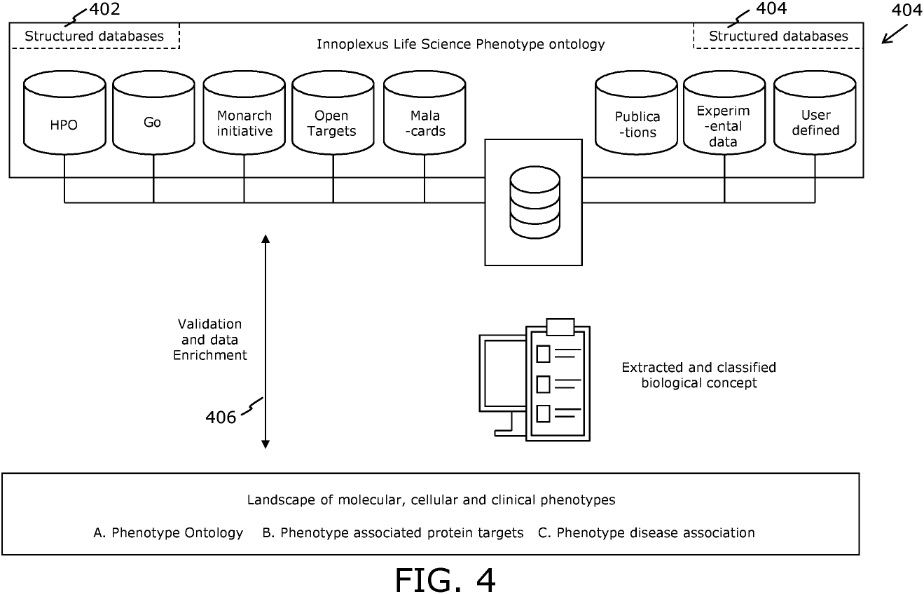

Referring to FIG. 4, there is shown a system 400 for determining phenotypes of at least one drug based on associations between the targets in the drug target list and the phenotypes, in accordance with the implementation of the present disclosure. Herein, structured databases 402 such as Human Phenotype Ontology (HPO), Gene Ontology (GO), Monarch Initiative, MalaCards and so forth may be used along with unstructured databases 404 such as publications, experimental data and/or user defined terminology. Subsequently, biological concepts are extracted and classified in the form of a landscape of molecular phenotype, cellular phenotype and clinical phenotypes, thereby deriving phenotype ontology, phenotype associated protein targets and phenotype disease association. The structured databases are communicably coupled 406 with the landscape of molecular phenotype, cellular phenotype and clinical phenotypes for validation and data enrichment.

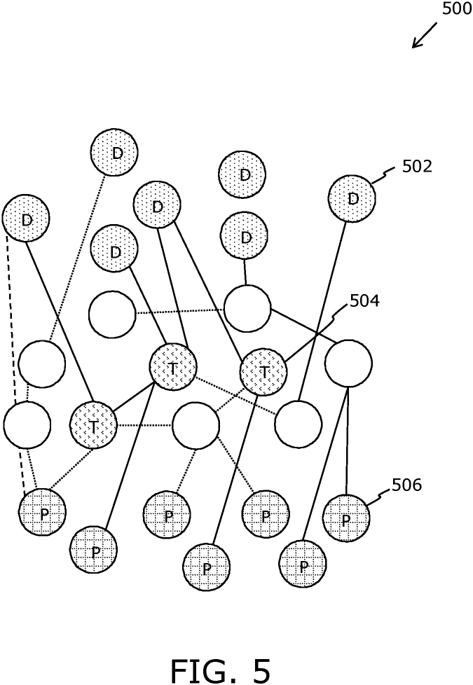

Referring to FIG. 5, there is shown a Drug-Target-Phenotype (DTP) network 500, in accordance with the embodiments of the present disclosure. Herein, the DTP network 500 comprises direct and indirect relation to at least one drug 502 with phenotypes 504 and phenotypes 506 of the at least one drug 502. Furthermore, the DTP network 500 are visually represented as simple graphs, with nodes and vertices, wherein the nodes have a number of edges attached to it. Herein, the drug is denoted by ‘D’, the phenotype by ‘T’ and the phenotype by ‘P’.

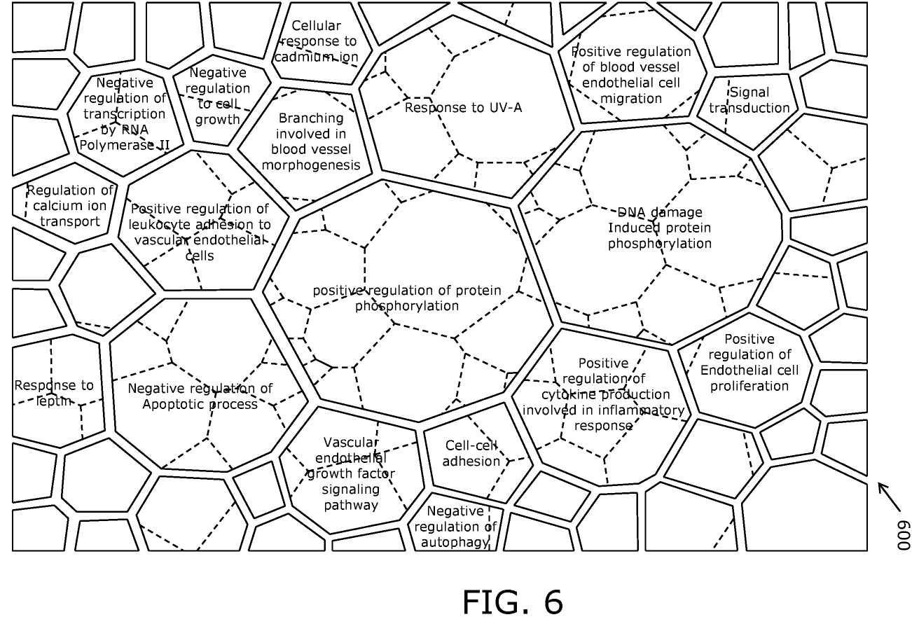

Referring to FIG. 6, there is shown a visual representation 600 to determine a parent level phenotype, in accordance with the embodiments of the present disclosure. Herein, the visual representation 600 is in the form of a bubble graph, wherein the parent level phenotypes are replaced with bubbles, and an additional dimension of the parent level phenotypes is represented in the size of the bubbles. For instance, the parent level phenotype maybe “positive regulation of protein phosphorylation”, “DNA damage induced protein phosphorylation”, “response to UV-A”, “positive regulation of cytokine production involved in inflammatory response”, “vascular endothelial growth factor signaling pathway” and so forth. Herein, the parent level phenotype of “positive regulation of protein phosphorylation” may comprise “positive regulation of transcription by RNA polymerase ii”, “regulation of transcription, DNA templated”, “positive regulation of transcription, DNA regulated” and so forth.

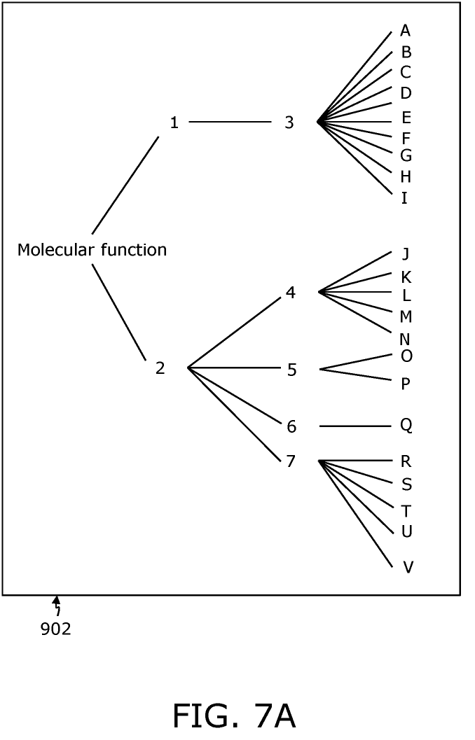

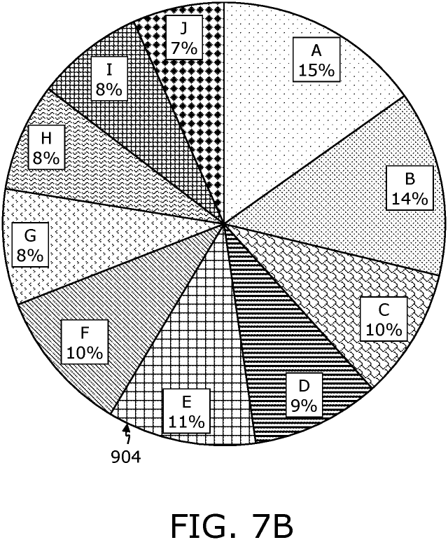

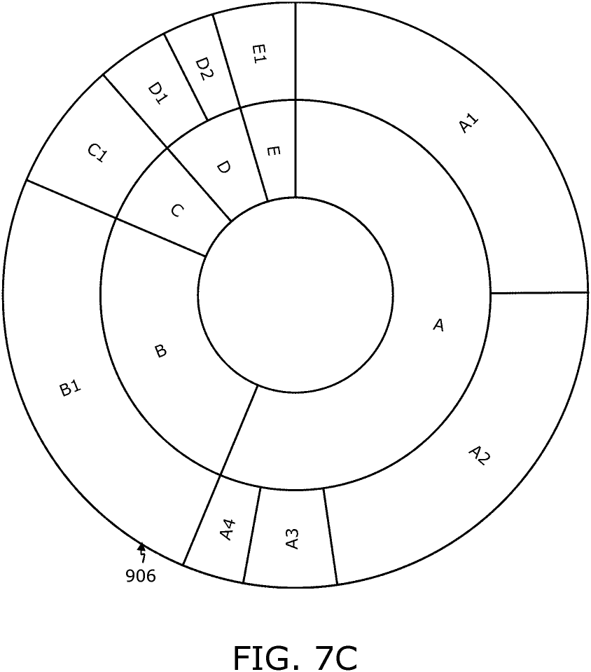

Referring to FIGS. 7A, 7B and 7C collectively, there is shown a visual representation of phenotypic signatures for a plurality of drugs, in accordance with the embodiments of the present disclosure. In FIG. 7A, there is shown an ontology tree 902 for a drug such as, “Erlotinib”, molecular function of the drug is divided into sub-paths, wherein the sub-paths may be “binding” and “catalytic activity”. Subsequently, the sub-path of “catalytic activity” may comprise different levels, such as “hydrolase activity”, “oxidoreductase activity” and so forth. The ontology tree 902 are labelled as shown in Table 3

TABLE 3

| | |
|---|---|
| LABEL | SUB-PATH |
| 1 | “binding” |
| 2 | “Catalytic activity” |
| 3 | “Protein binding” |
| 4 | “Catalytic activity, acting on a protein” |
| 5 | “Hydrolase activity” |
| 6 | “Oxidoreductase activity” |
| 7 | “Transferase activity” |
| A | “RNA polymerase II-specific DNA-binding transcription factor binding” |
| B | “Protease binding” |
| C | “Protein kinase binding” |
| D | “Protein phosphatase binding” |
| E | “Identical protein binding” |
| F | “Protein N-terminus binding” |
| G | “Enzyme binding” |
| H | “Transcription factor binding” |
| I | “Growth factor binding” |
| J | “Protein kinase activity” |
| K | “Peptidase activity” |
| L | “Protein tyrosine kinase activity” |
| M | “Protein serine/threonine kinase activity” |
| N | “Transmembrane receptor protein tyrosine kinase activity” |
| O | “ABC-type xenobiotic transporter activity” |
| P | “Peptidase activity” |
| Q | “Monooxygenase activity” |
| R | “Protein kinase activity” |
| S | “Protein tyrosine kinase activity” |
| T | “Protein serine/threonine kinase activity” |
| U | “Transmembrane receptor protein tyrosine kinase activity” |
| V | “Kinase activity” |

In FIG. 7B, there is shown a pie chart 904 to visualize parent level phenotype for a drug, such as “Erlotinib”. Herein, the pie chart 904 comprises the phenotypic signatures of parent level phenotypes, that may be for example, “transmembrane signaling receptor activity”, which may cover “15.2%” area on the pie chart 904, thereby depicting the percentage of the parent level phenotype hit by the drug. The labels for the pie chart 904 are as shown in Table 4

TABLE 4

| LABEL | PARENT LEVEL PHENOTYPE |
|---|---|
| A | “Transmembrane signaling receptor activity” |
| B | “Nitric-oxide synthase regulator activity” |
| C | “ATP binding” |
| D | “Growth factor binding” |
| E | “Protein phosphatase binding” |
| F | “Protein tyrosine kinase activity” |
| G | “Heme binding” |
| H | “Estrogen 2-hydroxylase activity” |
| I | “Caffeine oxidase activity” |
| J | “ABC-type xenobiotic transporter activity” |

In FIG. 7C, there is shown a sunburst chart 906 to visualize the phenotypic signatures of the parent level phenotypes and weightage of the phenotypes. Herein, the parent level phenotypes and the weightage of the phenotypes of the drug, such as “Erlotinib”, wherein the parent level phenotype, such as “catalytic activity” comprises the phenotypes along with the visual representation of the weightage, such as “transferase activity”, “catalytic activity, acting on a protein”, “hydrolase activity” and “oxidoreductase activity”. The labels for the sunburst chart 906 are as shown in Table 5

TABLE 5

| | |
|---|---|
| LABEL | PHENOTYPE |
| A | “Catalytic activity” |
| A1 | “Transferase activity” |
| A2 | “Catalytic activity, acting on a protein” |
| A3 | “Hydrolase activity” |
| A4 | “Oxidoreductase activity” |
| B | “Binding” |
| B1 | “Protein binding” |
| C | “Molecular transducer activity” |
| C1 | “Signaling receptor activity” |
| D | “Molecular function regulator” |
| D1 | “Enzyme regulator activity” |
| D2 | “Receptor regulator activity” |
| E | “Transporter activity” |
| E1 | “Transmembrane transporter activity” |

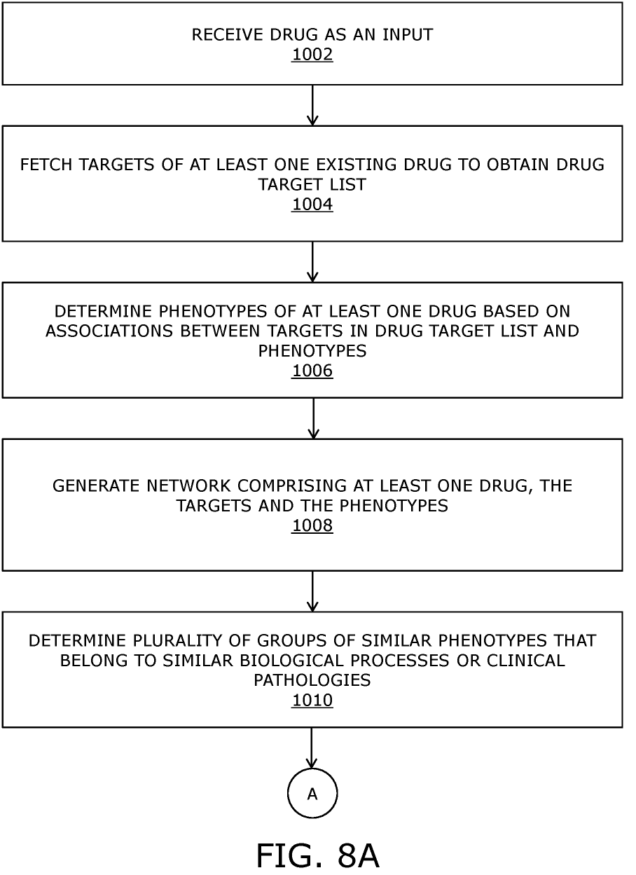

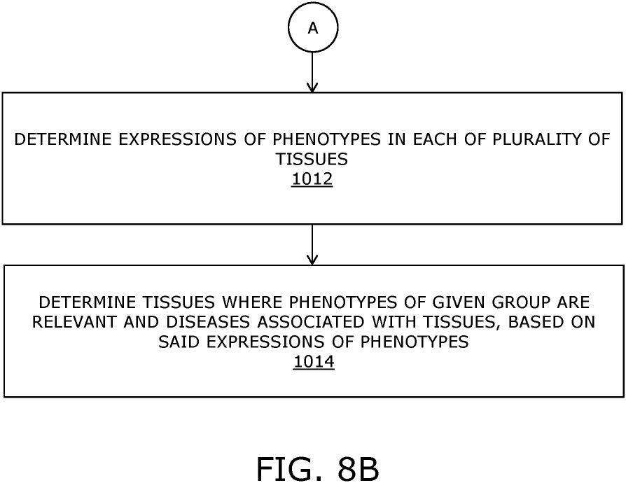

Referring to FIGS. 8A and 8B collectively illustrate a flowchart depicting steps of a method for enabling in-silico phenotypic screening of drugs, in accordance with the embodiments of the present disclosure. At step 1002, an input of a name of at least one drug is received. At step 1004, targets of at least one existing drug that is similar to the at least one drug to obtain a drug target list are fetched. At step 1006, phenotypes of the at least one drug based on associations between the targets in the drug target list and the phenotypes are determined, said associations being accessed from the phenotype ontological databank. At step 1008, a network comprising the at least one drug, the targets and the phenotypes is generated. At step 1010, a plurality of groups of similar phenotypes that belong to similar biological processes or clinical pathologies are determined. At step 1012, expressions of the phenotypes in each of a plurality of tissues, based on gene, protein and tissue expression data accessed from at least one database are determined. At step 1014, tissues where the phenotypes of a given group are relevant and diseases associated with the tissues are determined, based on said expressions of the phenotypes, thereby enabling phenotypic screening of the at least one drug.

Modifications to embodiments of the present disclosure described in the foregoing are possible without departing from the scope of the present disclosure as defined by the accompanying claims. Expressions such as “including”, “comprising”, “incorporating”, “have”, “is” used to describe and claim the present disclosure are intended to be construed in a non-exclusive manner, namely allowing for items, components or elements not explicitly described also to be present. Reference to the singular is also to be construed to relate to the plural.

1. A system for enabling in-silico phenotypic screening of drugs, the system is communicably coupled to
a phenotype ontological databank comprising information pertaining to a plurality of drugs and phenotypes corresponding to each of the plurality of drugs thereof;
wherein the system comprises a processor communicably coupled to a memory, the processor configured to
receive a name of at least one drug as an input;
fetch targets of at least one existing drug that is similar to the at least one drug to obtain a drug target list;
determine, phenotypes of the at least one drug based on associations between the targets in the drug target list and the phenotypes, said associations being accessed from the phenotype ontological databank;
generate a network comprising the at least one drug, the targets and the phenotypes;
determine a plurality of groups of similar phenotypes that belong to similar biological processes or clinical pathologies;
determine expressions of the phenotypes in each of a plurality of tissues, based on gene, protein and tissue expression data accessed from at least one database;
determine tissues where the phenotypes of a given group are relevant and diseases associated with the tissues, based on said expressions of the phenotypes, thereby enabling phenotypic screening of the at least one drug.

2. A system of claim 1, wherein the processor is configured to:
determine a corresponding weightage of each phenotype in a given group and a cumulative weightage of the phenotypes of the given group; and
generate a visual representation of the given group based on a ratio of individual weightages of the phenotypes of the given group to the cumulative weightage.

3. A system of claim 1, wherein the processor is configured to use literature mining to fetch the targets of the at least one existing drug that is similar to the at least one drug.

4. A system of claim 1, wherein the processor is configured to use a chemical similarity algorithm to identify the at least one existing drug that is similar to the at least one drug.

5. A system of claim 1, wherein the processor is configured to use a machine learning algorithm to predict targets of the at least one drug to obtain the drug target list, based on the targets of the at least one existing drug that is similar to the at least one drug.

6. A system of claim 1, wherein the processor is configured to use a molecular docking method to predict targets of the at least one drug to obtain the drug target list.

7. A system of claim 1, wherein the processor is configured to determine the phenotypes of the at least one drug by selecting only those phenotypes which are associated with at least two targets from the drug target list.

8. A computer-implemented method for enabling in-silico phenotypic screening of drugs, wherein the method is implemented using a system communicably coupled to a phenotype ontological databank comprising information pertaining to a plurality of drugs and phenotypes corresponding to each of the plurality of drugs thereof, the method comprising:
receiving a name of at least one drug as an input;
fetching targets of at least one existing drug that is similar to the at least one drug to obtain a drug target list;
determining, phenotypes of the at least one drug based on associations between the targets in the drug target list and the phenotypes, said associations being accessed from the phenotype ontological databank;
generating a network comprising the at least one drug, the targets and the phenotypes;
determining a plurality of groups of similar phenotypes that belong to similar biological processes or clinical pathologies;
determining expressions of the phenotypes in each of a plurality of tissues, based on gene, protein and tissue expression data accessed from at least one database;
determining tissues where the phenotypes of a given group are relevant and diseases associated with the tissues, based on said expressions of the phenotypes, thereby enabling phenotypic screening of the at least one drug.

9. A method of claim 8, further comprising:
determining a corresponding weightage of each phenotype in a given group and a cumulative weightage of the phenotypes of the given group; and
generating a visual representation of the given group based on a ratio of individual weightages of the phenotypes of the given group to the cumulative weightage.

10. A method of claim 8, further comprising using literature mining to fetch the targets of the at least one existing drug that is similar to the at least one drug.

11. A method of claim 8, further comprising using a chemical similarity algorithm to identify the at least one existing drug that is similar to the at least one drug.

12. A method of claim 8, further comprising using a machine learning algorithm to predict targets of the at least one drug to obtain the drug target list, based on the targets of the at least one existing drug that is similar to the at least one drug.

13. A method of claim 8, further comprising using a molecular docking method to predict targets of the at least one drug to obtain the drug target list.

14. A method of claim 8, further comprising determining the phenotypes of the at least one drug by selecting only those phenotypes which are associated with at least two targets from the drug target list.
